(19) United States
(12) Patent Application Publication
(10) Pub. No.: US 2006/0220007 A1
(43) Pub. Date: Oct. 5, 2006

(54) Acene compounds having a single terminal fused thiophene as semiconductor materials for thin film transistors and methods of making the same

(76) Inventors: David B. Bailey, Webster, NY (US); Xuan Mai, Deluth, GA (US); Andrea C. Scuderi, Rochester, NY (US); David H. Levy, Rochester, NY (US)

Correspondence Address: Paul A. Leipold;Patent Legal Staff, Eastman Kodak Company, 343 State Street, Rochester, NY, 14650-2201, US

(21) Appl. No.: 11/099,054

(22) Filed: Apr. 5, 2005

(51) Int. Cl. H01L 29/08; C07D 333/50

(52) U.S. Cl. 257/40; 549/41; 549/42

(57) ABSTRACT

A thin film transistor comprises a layer of organic semiconductor material comprising a comprising, in a thin film transistor, a thin film of organic semiconductor material that comprises an acene compound having a linear configuration of at least three fused benzene rings, which compound has, at one end only of the linear configuration, a terminal ring that is a fused substituted or unsubstituted thiophene, fused to an adjacent fused benzene ring of the acene compound.

### FIELD OF THE INVENTION

The present invention relates to the use of acene compounds containing a single terminal fused thiophene group as semiconductor materials in semiconductor films for thin film transistors. The invention relates to the use of these materials in thin film transistors for electronic devices and methods of making such transistors and devices.

### BACKGROUND OF THE INVENTION

Thin film transistors (TFTs) are widely used as a switching element in electronics, for example, in active-matrix liquid-crystal displays, smart cards, and a variety of other electronic devices and components thereof. The thin film transistor (TFT) is an example of a field effect transistor (FET). The best-known example of an FET is the MOSFET (Metal-Oxide-Semiconductor-FET), today's conventional switching element for high-speed applications. Presently, most thin film devices are made using amorphous silicon as the semiconductor. Amorphous silicon is a less expensive alternative to crystalline silicon. This fact is especially important for reducing the cost of transistors in large-area applications. Application of amorphous silicon is limited to low speed devices, however, since its maximum mobility (0.5-1.0 cm²/Vsec) is about a thousand times smaller than that of crystalline silicon.

Although amorphous silicon is less expensive than highly crystalline silicon for use in TFTs, amorphous silicon still has its drawbacks. The deposition of amorphous silicon, during the manufacture of transistors, requires relatively costly processes, such as plasma enhanced chemical vapor deposition and high temperatures (about 360° C.) to achieve the electrical characteristics sufficient for display applications. Such high processing temperatures disallow the use of substrates, for deposition, made of certain plastics that might otherwise be desirable for use in applications such as flexible displays.

In the past decade, organic materials have received attention as a potential alternative to inorganic materials such as amorphous silicon for use in semiconductor channels of TFTs. Organic semiconductor materials are simpler to process, especially those that are soluble in organic solvents and, therefore, capable of being applied to large areas by far less expensive processes, such as spin-coating, dip-coating and microcontact printing. Furthermore organic materials may be deposited at lower temperatures, opening up a wider range of substrate materials, including plastics, for flexible electronic devices. Accordingly, thin film transistors made of organic materials can be viewed as a potential key technology for plastic circuitry in display drivers, portable computers, pagers, memory elements in transaction cards, and identification tags, where ease of fabrication, mechanical flexibility, and/or moderate operating temperatures are important considerations.

Organic semiconductor materials can be used in TFTs to provide the switching and/or logic elements in electronic components, many of which require significant mobilities, well above 0.01 cm²/Vs, and current on/off ratios (hereinafter referred to as “on/off ratios”) greater than 1000. Organic TFTs having such properties are capable of use for electronic applications such as pixel drivers for displays and identification tags.

Organic materials for use as potential semiconductor channels in TFTs are disclosed, for example, in U.S. Pat. No. 5,347,144 to Garnier et al., entitled “Thin-Layer Field-Effect Transistors with MIS Structure Whose Insulator and Semiconductors Are Made of Organic Materials.”

An actively investigated area of organic semiconductors is the acene class of molecules. Acenes are compounds having at least three fused benzene rings in a linear configuration. Pentacene, having five fused benzene rings, is the mainstay of this class and has been demonstrated to achieve mobilities>1 cm²/Vs when vacuum deposited on selected surfaces (WO 03/041185 A2 to Kelly et al.). Pentacene has been extensively probed and modified in a search for improved performance, in particular for solubility, for organizational anchoring groups and for electronic modifications. Enhanced solubility of pentacene has been achieved by adding labile Diels-Alder adducts to the central ring (US 2003/0136964 A1) and by the addition of non-labile solubilizing groups (U.S. Pat. No. 6,690,029 to Anthony et al., issued Feb. 10, 2004). These and other strategies can enhance the solubility of pentacene. However, creating an ordered film from a disordered solution or from a vapor phase remains a challenge. C. Nuckolls et al (J. Am. Chem. Soc. 2004, 126, 15048-15050) recognized this dilemma and sought to functionalize one end of tetracene with methoxy or hydroxyl groups. These asymmetrically placed anchoring groups were intended to organize the molecule at the dielectric surface via hydrogen-bonding attraction. Other types of surface-molecule interactions beyond hydrogen bonding could be imagined for asymmetric type molecules.

The electronic and chemical properties (band gap, HOMO LUMO levels, oxidation potential) of the pentacene structure have been altered by for example replacing both of the terminal rings with thiophene rings (J. G. Laquindanum, H. E. Katz, A. J. Lovinger, J. Am. Chem. Soc. 1998, 120, 664-672). However, the placement of thiophenes at both ends of the acene inevitably leads to a cis-trans mixture. In addition, the unique directing effect of a single ended asymmetric structure is lost with a symmetrical approach.

Variations on the acene class have and continue to be widely investigated in order to maximize the semiconductor performance, stability, robustness, and to simplify the processability for enabling low cost manufacture.

### SUMMARY OF THE INVENTION

The present invention relates to the use of acenes containing a single terminal thiophene group as the semiconductor material in thin film transistors.

In particular, the present invention is directed to an article comprising, in a thin film transistor, a thin film of organic semiconductor material that comprises a compound comprising a linear configuration of at least three fused benzene rings, which compound has, at one end only of the linear configuration, a terminal ring that is a fused substituted or unsubstituted thiophene, fused on its b side to an adjacent fused benzene ring.

In one embodiment of the invention, the organic semiconductor material comprises an acene compound represented by the following structure I: 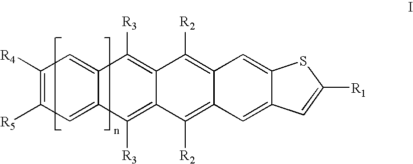
wherein n is an integer from 0 to 5, preferably 1 to 3, wherein both R₂ groups are the same and both R₃ groups are the same, at least one of R₂ or R₃ is hydrogen, and wherein R₂ or R₃ are independently selected from hydrogen, a branched or unbranched alkane having 2 to 18 carbon atoms, a branched or unbranched alkyl alcohol having 1 to 18 carbon atoms, a branched or unbranched alkene having 2 to 18 carbon atoms, a branched or unbranched alkyne having 2 to 18 carbon atoms, an aryl or heteroaryl (e.g. thiophene, pyridine) having 4 to 8 carbon atoms, an alkylaryl or alkyl-heteroaryl having 5 to 32 carbon atoms, which groups may be substituted or unsubstituted. R₁, R₄, and R₅ are independently selected from organic or inorganic groups that do not adversely affect the p-type semiconductor properties of the material.

The use of such compounds to can improve the stability, solution properties, film-forming characteristics, and processability of semiconductor films. In addition, the asymmetrically placed terminal thiophene ring in compounds of the present invention allows for facile introduction of a wide variety of performance-modifying end substituent R₁ groups on the terminal thiophene. In addition, the end substituent groups R₁ may contain added functionality that facilitates interaction with dielectric or conductor surfaces, enables intramolecular organization, enhances solubility in desirable coating solvents or imparts enhanced stability of the final device.

The present invention is also directed to a process for fabricating a thin film semiconductor device, comprising, not necessarily in the following order, the steps of:

(a) depositing, onto a substrate, a thin film of organic semiconductor material comprises a compound comprising a linear configuration of at least three fused benzene rings, which compound has, at one end only of the linear configuration, a terminal ring that is a fused substituted or unsubstituted thiophene, fused on its b side to an adjacent fused benzene ring, such that the thin film of organic semiconductor material exhibits a field effect electron mobility that is greater than 0.01 cm²/Vs;

(b) forming a spaced apart source electrode and drain electrode, wherein the source electrode and the drain electrode are separated by, and electrically connected with, the n-channel semiconductor film; and

(c) forming a gate electrode spaced apart from the semiconductor material.

Preferably, the compound is deposited on the substrate by sublimation or by solution-phase deposition, wherein the substrate has a temperature of no more than 100° C. during deposition.

The invention is also directed to an intermediate structure: 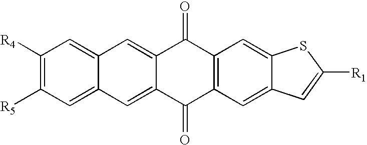
wherein R₁, R₄ and R₅ are as defined above.

Unless otherwise specifically stated, use of the term “substituted” or “substituent” means any group or atom other than hydrogen. Additionally, when the term “group” is used, it means that when a substituent group contains a substitutable hydrogen, it is also intended to encompass not only the substituent's unsubstituted form, but also its form to the extent it can be further substituted (up to the maximum possible number) with any substituent group or groups so long as the substituent does not destroy properties necessary for semiconductor utility. If desired, the substituents may themselves be further substituted one or more times with acceptable substituent groups. For example, an alkyl or alkoxy group can be substituted with one or more fluorine atoms. When a molecule may have two or more substituents, the substituents may be joined together to form an aliphatic or unsaturated ring such as a fused ring unless otherwise provided.

Examples of any of the alkyl groups mentioned above are methyl, ethyl, propyl, isopropyl, butyl, isobutyl, t-butyl, pentyl, hexyl, octyl, 2-ethylhexyl, and congeners. Alkyl or other organic groups preferably have 1 to 12 carbon atoms, more preferably 1 to 8 carbon atoms, most preferably 1 to 4 carbon atoms, and are intended to include branched or linear groups. Alkenyl groups, for example, can be vinyl, 1-propenyl, 1-butenyl, 2-butenyl, and congeners. Alkynl groups, for example, can be ethynyl, 1-propynyl, 1-butynyl, and congeners. Aryl groups, for example, can be phenyl, naphthyl, styryl, and congeners. Arylalkyl groups, for example, can be benzyl, phenethyl, and congeners.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical or analogous features that are common to the figures, and wherein:

FIG. 1 illustrates a cross-sectional view of a typical organic thin film transistor having a bottom contact configuration; and

FIG. 2 illustrates a cross-sectional view of a typical organic thin film transistor having a top contact configuration.

### DESCRIPTION OF THE INVENTION

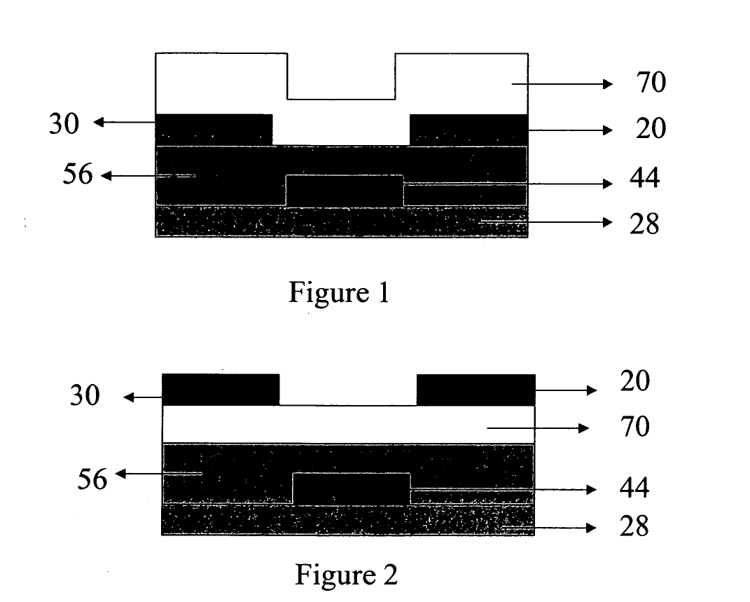

Cross-sectional views of a typical organic thin film transistor are shown in FIGS. 1 and 2, wherein FIG. 1 illustrates a typical bottom contact configuration and FIG. 2 illustrates a typical top contact configuration.

Each thin film transistor (TFT) in FIGS. 1 and 2 contains a source electrode 20, a drain electrode 30, a gate electrode 44, a gate dielectric 56, a substrate 28, and the semiconductor 70 of the invention in the form of a film connecting the source electrode 20 to drain electrode 30, which semiconductor comprises a compound selected from the class of compounds based on a fused acene containing a terminal thiophene group described herein.

When the TFT operates in an accumulation mode, the charges injected from the source 20 into the semiconductor are mobile and a current flows from source to drain, mainly in a thin channel region within about 100 Angstroms of the semiconductor-dielectric interface. See A. a Dodabalapur, L. Torsi H. E. Katz, Science 1995, 268, 270, hereby incorporated by reference. In the configuration of FIG. 1, the charge need only be injected laterally from the source 20 to form the channel. In the absence of a gate field the channel ideally has few charge carriers; as a result there is ideally no source-drain conduction.

The off current is defined as the current flowing between the source electrode 20 and the drain electrode 30 when charge has not been intentionally injected into the channel by the application of a gate voltage. For a p-channel accumulation-mode TFT such as is typical for many organic semiconductors, the off behavior occurs for a gate-source voltage more positive than a certain voltage known as the threshold voltage. See Sze in Semiconductor Devices—Physics and Technology, John Wiley & Sons (1981), pages 438-443. The on current is defined as the current flowing between the source 20 and the drain 30 when charge carriers have been accumulated intentionally in the channel by application of an appropriate voltage to the gate electrode, and the channel is conducting. For a p-channel accumulation-mode TFT, this occurs at gate-source voltages more negative than the threshold voltage. It is desirable for this threshold voltage to be zero, or slightly negative, for n-channel operation. This ensures that when the gate is held at ground along with the source, the device is in the off mode. Switching between on and off is accomplished by the application and removal of an electric field from the gate electrode 44 across the gate dielectric 56 to the semiconductor-dielectric interface, effectively charging a capacitor.

In accordance with the invention, the organic semiconductor materials used in the present invention can exhibit high performance under ambient conditions without the need for special chemical underlayers.

The semiconductor film of the present invention, comprising acene compounds containing a terminal thiophene group as described herein is capable of exhibiting field effect electron mobility greater than 10⁻⁶ cm²/Vs and preferably greater than 0.01 cm²/Vs.

In addition, the semiconductor film of the invention is capable of providing on/off ratios of at least 10², advantageously at least 10⁵. The on/off ratio is the ratio of the maximum to the minimum drain current as the gate voltage is varied from 5 to −50V, and employing a silicon dioxide gate dielectric.

As indicated above, the invention is directed to an article comprising a thin film of an organic semiconductor material that comprises an acene compound represented by the following structure I: 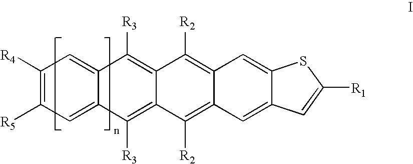
wherein n is an integer from 0 to 5, preferably 1 to 3, most preferably 2 (i.e., defining a pentacene nucleus fused to a thiophene), at least one of R₂ or R₃ is hydrogen, and R₂ or R₃ is selected from hydrogen, a branched or unbranched alkane having 2 to 18 carbon atoms, a branched or unbranched alkyl alcohol having 1 to 18 carbon atoms, a branched or unbranched alkene having 2 to 18 carbon atoms, a branched or unbranched alkyne having 2 to 18 carbon atoms, an aryl or heteroaryl (e.g. thiophene, pyridine) having 4 to 8 carbon atoms, an alkylaryl or alkyl-heteroaryl having 5 to 32 carbon atoms, all of which groups other than hydrogen can be substituted or unsubstituted.

In a preferred embodiment, R₂ is a —C≡C—R₆ alkyne group in which R₆ is any of the groups mentioned for R₂ (except another alkyne) or a Si(R₇)₃ group where the R₇ group is independently a branched or unbranched alkane having 1 to 10 carbon atoms, a branched or unbranched alkyl alcohol having 1 to 10 carbon atoms, or a branched or unbranched alkene having 2 to 10 carbon atoms.

R₁, R₄, and R₅ are independently selected from hydrogen and organic or inorganic groups that do not adversely affect the p-type semiconductor properties of the material.

Preferably, R₄ and R₅ are groups that are independently selected from the group consisting of hydrogen, electron-donating substituents (for example, alkyl, alkenyl, alkynyl, alkoxy, or thioalkoxy), aryl, substituted aryl, halogen substituents (e.g., fluorine), and combinations thereof, wherein R₄ and R₅ together can form a non-aromatic ring when on adjacent carbon atoms. As used herein, the term “combinations” of substituents includes, monovalent combinations (for example, a bromomethyl substituent) as well as substituents formed by the bonding together of the substituents on two adjacent carbon atoms to form a ring structure (for example, two alkyl substituents on adjacent carbon atoms can be bonded together to form a divalent alkylene group that bridges or links the carbon atoms).

More preferably, R₄ and R₅ are groups are independently selected from the group consisting of hydrogen, alkyl groups, alkoxy groups, thioalkoxy groups, halogen atoms, and combinations thereof. Even more preferably, each substituent is independently hydrogen, an alkyl group, an alkoxy group, or a combination thereof. Preferred alkyl groups are methyl, n-hexyl, n-nonyl, n-dodecyl, sec-butyl, 3,5,5-trimethylhexyl, 2-ethylhexyl, or a hydrogen atom.

R₁ can include any of the R₄, R₅ groups as well as a wide variety of other groups, including, organic groups containing an alcohol, phenol, thiol, carboxylic acid, amide, or carbamate functionality, or the like, to achieve a hydrogen bonding interaction. Or the R₁ group may achieve hydrophobic interactions by means of an alkyl, aryl, perfluoroalkyl, perfluoroaryl, or siloxane functionality, or the like. R₁ may also contain a reactive functionality such as trichlorosilane, trialkoxysilane, an acid chloride, the N-hydroxysuccinimide ester of a carboxylic acid, or the like. for reaction with a surface adjacent to the organic semiconductor material. A preferred R₁ group is a short-chain C1 to 6 alkyl group substituted with a functionality comprising at least one hydroxyl or carbonyl group.

In one embodiment, R₂ and R₃ are both hydrogen in Structure I above. In another embodiment, only one of R₂ and R₃ are hydrogen. In yet another embodiment, R₄ and R₅ are hydrogen.

A preferred class of compounds is the organic semiconductor materials that comprise a compound that is an acene compound represented by the following Structure II: 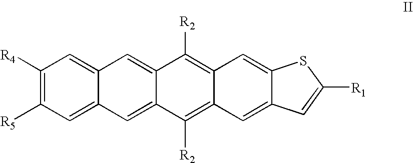
wherein R₁, R₂, R₄ and R₅ are as defined above. In one preferred embodiment of the invention, R₂ is selected from the group consisting of the following:
—C≡C—R₆ where R₆=H or alkyl or aryl
—C≡C—Si(R₇)₃ where R₇=alkyl.

In one embodiment, R₂, R₄ and R₅ are hydrogen in Structure II. In another embodiment, R₂ is not hydrogen. When R₂ is not hydrogen, a preferred class of compounds is represented by the following structure III: 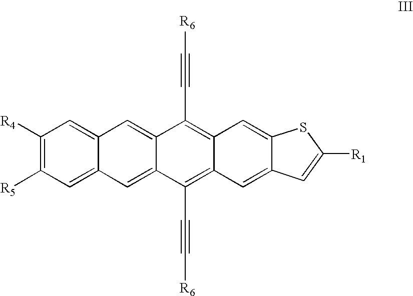
wherein both R₆ groups are the same, R₁, R₄, and R₅ are as defined previously, and R₆ is a branched or unbranched alkane having 2 to 18 carbon atoms, a branched or unbranched alkyl alcohol having 1 to 18 carbon atoms, an aryl or heteroaryl (e.g. thiophene, pyridine) having 4 to 8 carbon atoms, an alkylaryl or alkyl-heteroaryl having 5 to 32 carbon atoms or a hydrogen, or a Si(R₇)₃ group where the R₇ group is independently a branched or unbranched alkane having 1 to 10 carbon atoms, a branched or unbranched alkyl alcohol having 1 to 10 carbon atoms, or a branched or unbranched alkene having 2 to 10 carbon atoms.

In one embodiment, R₄ and R₅ are hydrogen in the above Structure III. Representative examples of acenes containing a terminal thiophene group are the following:

| | |
|---|---|
| SC-1 | 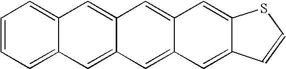 |
| SC-2 | 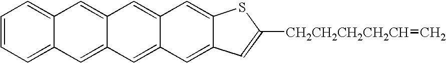 |
| SC-3 | 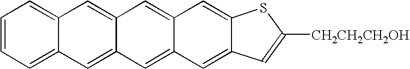 |
| SC-4 | 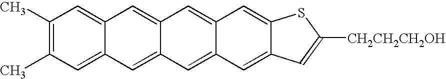 |
| SC-5 | 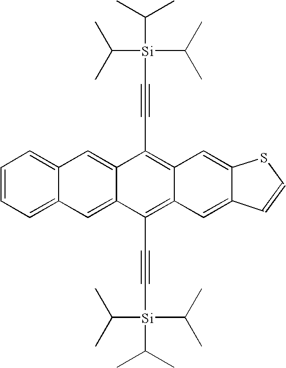 |
| SC-6 | 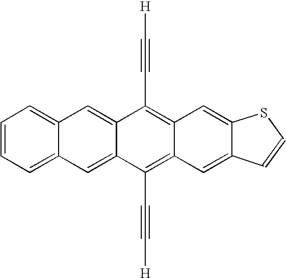 |
| SC-7 | 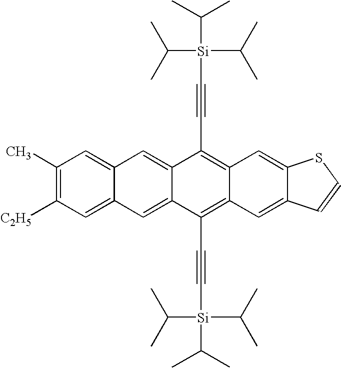 |
| SC-8 | 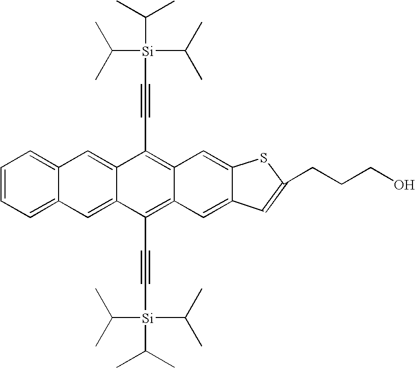 |
| SC-9 | 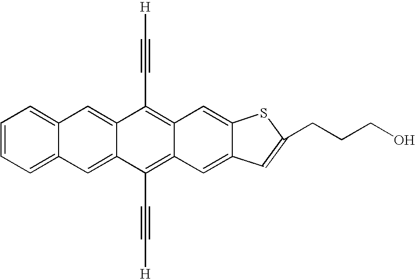 |
| SC-10 | 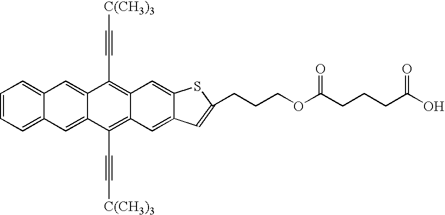 |
| SC-11 | 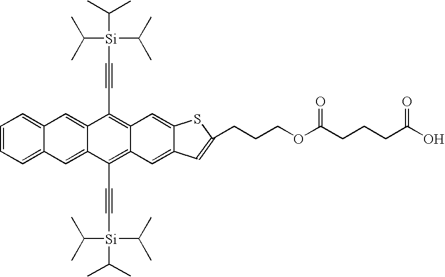 |
| SC-12 | 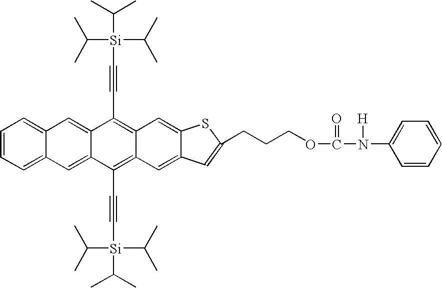 |
| SC-13 | 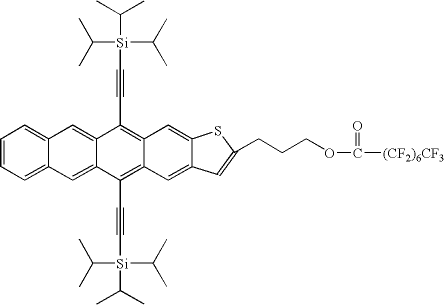 |
| SC-14 | 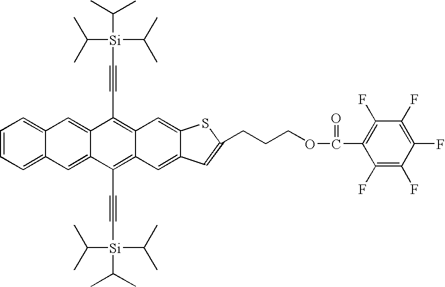 |

The compounds of the present invention may be prepared by reaction of a suitable dialdehyde with the appropriate dione, as exemplified below. An alternative synthetic approach is disclosed in US Patent 2003/0105365 to Smith et al., wherein an anhydride is reacted with the appropriate aromatic compound.

Another aspect of the invention relates to process for the production of semiconductor components and electronic devices incorporates such components. In one embodiment, a substrate is provided and a layer of the semiconductor material as described above can be applied to the substrate, electrical contacts being made with the layer. The exact process sequence is determined by the structure of the desired semiconductor component. Thus, in the production of an organic field effect transistor, for example, a gate electrode can be first deposited on a flexible substrate, for example an organic polymer film, the gate electrode can then be insulated with a dielectric and then source and drain electrodes and a layer of the semiconductor material can be applied on top. The structure of such a transistor and hence the sequence of its production can be varied in the customary manner known to a person skilled in the art. Thus, alternatively, a gate electrode can be deposited first, followed by a gate dielectric, then the organic semiconductor can be applied, and finally the contacts for the source electrode and drain electrode deposited on the semiconductor layer. A third structure could have the source and drain electrodes deposited first, then the organic semiconductor, with dielectric and gate electrode deposited on top.

In yet another embodiment of the present invention, source drain and gate can all be on a common substrate and the gate dielectric can enclose gate electrode such that gate electrode is electrically insulated from source electrode and drain electrode, and the semiconductor layer can be positioned over the source, drain and dielectric.

The skilled artisan will recognize other structures can be constructed and/or intermediate surface modifying layers can be interposed between the above-described components of the thin film transistor.

A support can be used for supporting the OTFT during manufacturing, testing, and/or use. The skilled artisan will appreciate that a support selected for commercial embodiments may be different from one selected for testing or screening various embodiments. In some embodiments, the support does not provide any necessary electrical function for the TFT. This type of support is termed a “non-participating support” in this document. Useful materials can include organic or inorganic materials. For example, the support may comprise inorganic glasses, ceramic foils, polymeric materials, filled polymeric materials, coated metallic foils, acrylics, epoxies, polyamides, polycarbonates, polyimides, polyketones, poly(oxy-1,4-phenyleneoxy-1,4-phenylenecarbonyl-1,4-phenylene) (sometimes referred to as poly(ether ether ketone) or PEEK), polynorbornenes, polyphenyleneoxides, poly(ethylene naphthalenedicarboxylate) (PEN), poly(ethylene terephthalate) (PET), poly(phenylene sulfide) (PPS), and fiber-reinforced plastics (FRP).

A flexible support is used in some embodiments of the present invention. This allows for roll processing, which may be continuous, providing economy of scale and economy of manufacturing over flat and/or rigid supports. The flexible support chosen preferably is capable of wrapping around the circumference of a cylinder of less than about 50 cm diameter, more preferably 25 cm diameter, most preferably 10 cm diameter, without distorting or breaking, using low force as by unaided hands. The preferred flexible support may be rolled upon itself.

In some embodiments of the invention, the support is optional. For example, in a top construction as in FIG. 2, when the gate electrode and/or gate dielectric provides sufficient support for the intended use of the resultant TFT, the support is not required. In addition, the support may be combined with a temporary support. In such an embodiment, a support may be detachably adhered or mechanically affixed to the support, such as when the support is desired for a temporary purpose, e.g., manufacturing, transport, testing, and/or storage. For example, a flexible polymeric support may be adhered to a rigid glass support, which support could be removed.

The gate electrode can be any useful conductive material. A variety of gate materials known in the art, are also suitable, including metals, degenerately doped semiconductors, conducting polymers, and printable materials such as carbon ink or silver-epoxy. For example, the gate electrode may comprise doped silicon, or a metal, such as aluminum, chromium, gold, silver, nickel, palladium, platinum, tantalum, and titanium. Conductive polymers also can be used, for example polyaniline, poly(3,4-ethylenedioxythiophene)/poly(styrene sulfonate) (PEDOT:PSS). In addition, alloys, combinations, and multilayers of these materials may be useful.

In some embodiments of the invention, the same material can provide the gate electrode function and also provide the support function of the support. For example, doped silicon can function as the gate electrode and support the OTFT.

The gate dielectric is provided on the gate electrode. This gate dielectric electrically insulates the gate electrode from the balance of the OTFT device. Thus, the gate dielectric comprises an electrically insulating material. The gate dielectric should have a dielectric constant above about 2, more preferably above about 5. The dielectric constant of the gate dielectric also can be very high if desired, for example, 80 to 100 or even higher. Useful materials for the gate dielectric may comprise, for example, an inorganic electrically insulating material. The gate dielectric may comprise a polymeric material, such as polyvinylidenedifluoride (PVDF), cyanocelluloses, polyimides, etc.

Specific examples of materials useful for the gate dielectric include strontiates, tantalates, titanates, zirconates, aluminum oxides, silicon oxides, tantalum oxides, titanium oxides, silicon nitrides, barium titanate, barium strontium titanate, barium zirconate titanate, zinc selenide, and zinc sulfide. In addition, alloys, combinations, and multilayers of these examples can be used for the gate dielectric. Of these materials, aluminum oxides, silicon oxides, and zinc selenide are preferred. In addition, polymeric materials such as polyimides, and insulators that exhibit a high dielectric constant. Such insulators are discussed in U.S. Pat. No. 5,981,970 hereby incorporated by reference.

The gate dielectric can be provided in the OTFT as a separate layer, or formed on the gate such as by oxidizing the gate material to form the gate dielectric. The dielectric layer may comprise two or more layers having different dielectric constants.

The source electrode and drain electrode are separated from the gate electrode by the gate dielectric, while the organic semiconductor layer can be over or under the source electrode and drain electrode. The source and drain electrodes can be any useful conductive material. Useful materials include most of those materials described above for the gate electrode, for example, aluminum, barium, calcium, chromium, gold, silver, nickel, palladium, platinum, titanium, polyaniline, PEDOT:PSS, other conducting polymers, alloys thereof, combinations thereof, and multilayers thereof.

The thin film electrodes (e.g., gate electrode, source electrode, and drain electrode) can be provided by any useful means such as physical vapor deposition (e.g., thermal evaporation, sputtering) or ink jet printing. The patterning of these electrodes can be accomplished by known methods such as shadow masking, additive photolithography, subtractive photolithography, printing, microcontact printing, and pattern coating.

The organic semiconductor layer can be provided over or under the source and drain electrodes, as described above in reference to the thin film transistor article. The present invention also provides an integrated circuit comprising a plurality of OTFTs made by the process described herein. The semiconductor material made using the above compounds based on a fused acene containing a terminal thiophene group are capable of being formed on any suitable substrate which can comprise the support and any intermediate layers such as a dielectric or insulator material, including those known in the art.

The entire process of making the thin film transistor or integrated circuit of the present invention can be carried out below a maximum support temperature of about 450° C., preferably below about 250° C., more preferably below about 150° C., and even more preferably below about 100° C., or even at temperatures around room temperature (about 25° C. to 70° C.). The temperature selection generally depends on the support and processing parameters known in the art, once one is armed with the knowledge of the present invention contained herein. These temperatures are well below traditional integrated circuit and semiconductor processing temperatures, which enables the use of any of a variety of relatively inexpensive supports, such as flexible polymeric supports. Thus, the invention enables production of relatively inexpensive integrated circuits containing organic thin film transistors with significantly improved performance.

Compounds used in the invention can be readily processed and are thermally stable to such as extent that they can be vaporized. The compounds possess significant volatility, so that vapor phase deposition, where desired, is readily achieved. Such compounds can be deposited onto substrates by vacuum sublimation or by solvent processing, including dip coating, drop casting, spin coating, blade coating.

Deposition by a rapid sublimation method is also possible. One such method is to apply a vacuum of 35 mtorr to a chamber containing a substrate and a source vessel that holds the compound in powdered form, and heat the vessel over several minutes until the compound sublimes onto the substrate. Generally, the most useful compounds form well-ordered films, with amorphous films being less useful.

Alternatively, for example, the compounds described above can first be dissolved in a solvent prior to spin-coating or printing for deposition on a substrate.

Devices in which the semiconductor films of the invention are useful include especially thin film transistors (TFTs), especially organic field effect thin-film transistors. Also, such films can be used in various types of devices having organic p-n junctions, such as described on pages 13 to 15 of US 2004,0021204 A1 to Liu, which patent is hereby incorporated by reference.

Electronic devices in which TFTs and other devices are useful include, for example, more complex circuits, e.g., shift registers, integrated circuits, logic circuits, smart cards, memory devices, radio-frequency identification tags, backplanes for active matrix displays, active-matrix displays (e.g. liquid crystal or OLED), solar cells, ring oscillators, and complementary circuits, such as inverter circuits, for example, in combination with other transistors made using available n-type organic semiconductor materials. In an active matrix display, a transistor according to the present invention can be used as part of voltage hold circuitry of a pixel of the display. In devices containing the TFTs of the present invention, such TFTs are operatively connected by means known in the art. The present invention further provides a method of making any of the electronic devices described above. Thus, the present invention is embodied in an article that comprises one or more of the TFTs described.

Advantages of the invention will be demonstrated by the following examples, which are intended to be exemplary.

A. Materials

A semiconductor film comprising the following Compound I, an acene compound containing a terminal thiophene substituted with a hexenyl group, was prepared as follows.

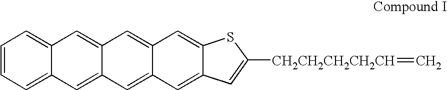

The compound 5-hexenyl-2,3-thiophenedicarboxaldehyde was prepared as follows: 2,3-Thiophenedicarboxaldehyde was protected as the bisacetal with ethylene glycol as described by Katz (H. E Katz et al, J. Am Chem. Soc, vol. 120, 1998, 667). The bisacetal (7.0 g, 31 mmole) was dissolved in 108 g of anhydrous THF in a 250 ml flask. The solution was purged with argon, cooled to −78 C and then treated with a solution of n-butyllitium (1.6M, 25 ml, 40 mmole) to give a thick yellow mixture. 6-Bromo-1-hexene (6.6 g, 40 mmole) was added after 10 minutes. The mixture was allowed to warm to room temperature and was then heated to 50 C for 3 hours. Gas chromatography indicated a 3:1 mixture of hexenyl substituted product to unreacted starting material. The product was purified by column chromatography on silica gel using a 20:80 ether:ligroin mixture to yield 5.62 g (58%) of 2,3-bis(1,3-dioxolan-2-yl)-5-hexenylthiophene. The acetal groups were deblocked by dissolving the 5.62 g (18 mmole) in a solution of THF (84 g), water (65 g) and 10N hydrochloric acid (5 ml) and heating at 60 C for 3 hours. The solution was added to 200 ml of ether, washed twice with water, dried with magnesium sulfate, filtered, concentrated and purified by column chromatography using a 10:90 mixture of ether:ligroin. The yield was 2.47 g (61%) of 95% pure (by gas chromatography) 5-hexenyl-2,3-thiophenedicarboxaldehyde.

The compound 2,3-dihydro-1,4-anthraquinone was prepared as follows: A solution of phthalic dicarboxaldehyde (5.25 g, 39 mmole), excess of 1,4-cyclohexanedione (17.6 g, 157 mmole), methanol (65 ml) and triethylamine (1 g, 10 mmole) were heated at 50 C for 20 hours under argon in a 250 ml flask. The large excess of cyclohexanedione was to minimize double substitution of phthalic dicarboxaldehyde on the cyclohexanedione. The methanol was distilled from the mixture, a high boiling chaser solvent, tetraethyleneglycol dimethylether (TEGDME, 30 g), was added and the mixture was distilled to near dryness under high vacuum at 135 C. TEGDME (10 g) was added, the mixture was dissolved in 150 ml of argon purged ether, washed twice with 150 ml of argon purged water to remove as much cyclohexanedione as possible. The yield of 2,3-dihydro-1,4-anthraquinone after removal of the ether was 2.65 g (32%). The product contained 10% 1,4-anthraquinone side product as measured by gas chromatography.

Tetracenehexenylthiophenequinone was prepared as follows: A 250 ml flask was charged with 2,3-dihydro-1,4-anthraquinone (2.5 g, 11.9 mmole), 5-hexenyl-2,3-thiophenedicarboxaldehyde (2.64 g, 11.9 mmole), methanol (65 g) and triethylamine (0.3 g, 3 mmole). The mixture was heated under argon at 50 C for 24 hours. The product was precipitated with water, isolated by filtration, and then purified by column chromatography with an 80:20 mixture of ligroin:methylene chloride. The yield was 2.72 g (58%) of a bright yellow powder of 92% purity.

The quinone was reduced to hexenyl substituted tetracenethiophene as follows: A mixture of aluminum wire (3.21 g, 119 mmole), mercury dichloride (0.06 g, 0.24 mmole), carbon tetrachloride (0.91 g, 6 mmole), cyclohexanol (100 g) were heated at reflux for 24 hours to form a clear solution. The diketone (2.5 g, 9.9 mmole) was added and the mixture was heated at reflux for 2 days. The reaction mixture was added to 50:50 water:acetic acid, the precipitate was isolated by filtration, mixed with 1N hydrochloric acid for 3 hours, and isolated by filtration to give 1.98 g of a red solid. A 0.5 g sample was sublimed to give 80 mg of product. A semiconductor film comprising the following Compound II, an acene compound containing a pendant triisopropylsilylacetylenes and a terminal thiophene group, was prepared as follows.

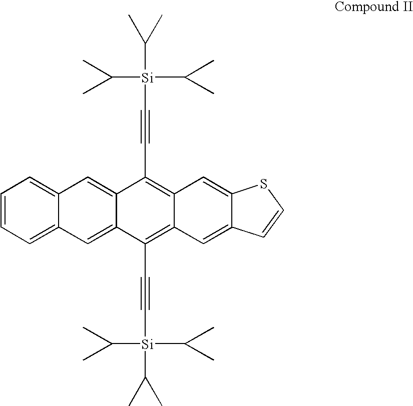

A mixture of 2,3-dihydro-1,4-anthraquinone as described above (3.46 g, 16.5 mmole), 2,3-thiophenedicarboxaldehyde (3.46 g, 24.7 mmole), methanol (88 g) and triethylamine (0.42 g, 4.1 mmole) was heated at 50 C for 24 hours. The precipitate was filtered, washed with methanol, purified by column chromatography with 60:40 methylene chloride:ligroin to give 2.5 g (48%) of product. The tetracenequinone containing a terminal thiophene (1.2 g, 3.8 mmole) was added to a solution of triisopropylsiliylacetylene (3.83 g, 21 mmole) in anhydrous THF (72 ml) that had been treated at −78 C with 1.6M butyllithium (14.3 ml, 22.9 mmole) and warmed to room temperature for 1 hour. The solution was stirred for 24 hrs, quenched with 2 ml water, filter, and stripped of solvent. Tin dichloride (2.9 g, 15.3 mmole), and THF were added to from a deep red solution that was stirred for 1 hr. The product was extract with ether, wash with 1N hydrochloric acid, washed three times with water, the isolated solid was purified by column chromatography with hexane, recrystallized from acetone and then from hexane to yield dark crystals. HPLC analysis showed 96% bis(triisopropylsilylethynyl)tetracenethiophene.

A semiconductor film comprising the following Compound III, an acene compound containing pendant triisopropylsilylacetylenes and a terminal thiophene substituted with a 3-hydroxypropyl group, was prepared as follows.

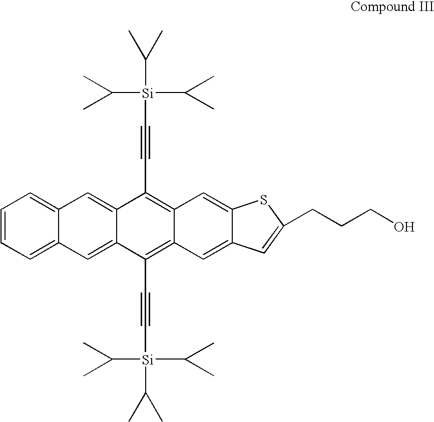

The compound 2,3-thiophenedicarboxaldehyde was protected as the bisacetal with ethylene glycol as described in compound I, substituted with 2-(3-bromopropoxy)tetrahydro-2H-pyran and deblocked by the same procedure as used for 6-bromo-1-hexene described in compound I and then purified by column chromatography using a mixture of ether:ligroin to give 3-hydroxypropyl-2,3-thiophenedicarboxaldehyde. The same procedure in Compound I was used to prepare tetracene 3-hydroxypropylthiophenequinone. The hydroxyl group was blocked by reaction of 1.7 g (4.5 mmole) with 3,4-dihydro-2H-pyran (12 g, 142 mmole), p-toluenesulfonic acid (0.02 g, 0.2 mmole) in dioxane (14 g). The mixture was heated to 90 C for 10 minutes to form a solution, cooled to room temperature, treated with 5 drops of triethylamine and 20 ml of ether. The mixture was filtered to yield 1.57 g of orange solid. The reaction with triisopropylsiliylacetylene and the subsequent reaction with tin dichloride were carried as in the preparation of compound II. The pyran group was removed by treating 0.89 g in 30 ml of THF with 1 ml of water, 2 ml of methanol, 0.04 g p-toluenesulfonic acid, and heating at 60 C for 3 hours. The solution was treated with triethylamine and purified by chromatography with ligroin and then chromatographed a second time with 10:90 ether:ligr to yield 0.52 g of bis(triisopropylsilylethynyl)tetracene 3-hydroxypropylthiophene. The product was recrystallized from acetone twice and then from hexanes once.

B. Device Preparation

In order to test the electrical characteristics of the various materials of this invention, field-effect transistors were typically made using the top-contact geometry. The substrate used is a heavily doped silicon wafer, which also serves as the gate of the transistor. The gate dielectric is a thermally grown SiO₂ layer with a thickness of 215 nm.

### EXAMPLE I

The active layer of Structure I was deposited via vacuum deposition in a thermal evaporator. A heavily doped silicon wafer with a thermally grown SiO₂ layer with a thickness of 165 nm was used as the substrate. The wafer was cleaned for 10 minutes in a piranha solution, followed by a 6-minute exposure in an UV/ozone chamber. The cleaned surface was then treated with a thin layer of polystyrene by spin coating.

The purified semiconducting material was deposited by vacuum sublimation at a pressure of 5×10⁻⁷ Torr and a rate of 0.5 Angstroms per second to a thickness of 40 nm as measured by a quartz crystal. During deposition the substrate was held at a constant temperature of 60° C. The sample was exposed to air for a short time prior to subsequent deposition of Ag source and drain electrodes through a shadow mask to a thickness of 50 nm. The devices made had a 500 micron channel width, with channel lengths varying from 20-80 microns. The mobility was 5.38×10⁻³ cm²/V-s, and the on/off ratio was 2.0×10³.

### EXAMPLE II A AND II B

The active layer of Structure II was deposited via spin coating at 1200 rpm from a 0.2 wt % solution in chlorobenzene (A) or fluorobenzene (B). A heavily doped silicon wafer with a thermally grown SiO₂ layer with a thickness of 215 nm was used as the substrate. The wafer was cleaned for 10 minutes in a piranha solution, followed by a 6-minute exposure in an UV/ozone chamber. The surface had no further treatments. Immediately following semiconductor deposition, Au source and drain electrodes were evaporated through a shadow mask to a thickness of 50 nm. The devices made had a 500 micron channel width, with channel lengths varying from 20-100 microns. Devices spun from chlorobenzene resulted in mobilities of 5.0×10⁻¹ cm²/N-s, and the on/off ratio was 2.9×10⁵. Devices spun from fluorobenzene resulted in mobilities of 8.0×10⁻² cm² V-s, and the on/off ratio was 2.10×10⁵.

### EXAMPLE III A AND III B

The active layer of Structure III was deposited via spin coating at 1200 rpm from a 0.2 wt % solution in isopropyl alcohol (A) or chlorobenzene (B). A heavily doped silicon wafer with a thermally grown SiO₂ layer with a thickness of 215 nm was used as the substrate. The wafer was cleaned for 10 minutes in a piranha solution, followed by a 6-minute exposure in an UV/ozone chamber. The surface had no further treatments. Immediately following semiconductor deposition, Au source and drain electrodes were evaporated through a shadow mask to a thickness of 50 nm. The devices made had a 500 micron channel width, with channel lengths varying from 20-100 microns. Devices spun from isopropyl alcohol resulted in a maximum mobility of 1.08×10⁻⁴ cm²/V-s, with an on/off ratio of 9.40×10². Devices spun from chlorobenzene resulted in a mobility of 6.0×10⁻⁶ cm²/V-s, with an on/off ratio of 5.5×10².

C. Device Measurement and Analysis

Electrical characterization of the fabricated devices was performed with a Hewlett Packard HP 4145b® parameter analyzer. The probe measurement station was held in a positive N₂ environment for all measurements with the exception of those purposely testing the stability of the devices in air. The measurements were performed under sulfur lighting unless sensitivity to white light was being investigated. The devices were exposed to air prior to testing.

For each device, the drain current (Id) was measured as a function of source-drain voltage (Vd) for various values of gate voltage (Vg). For most devices, Vd was swept from 0 to −50 V for each of the gate voltages measured. The gate voltage for this measurement was typically stepped from 0 to −50V in increments of 10V. In these measurements, the gate current (Ig) was also recorded in to detect any leakage current through the device. Furthermore, for each device the drain current was measured as a function of gate voltage for various values of source-drain voltage. For most devices, Vg was swept from 5 V to −50 V for each of the drain voltages measured, typically −30 V, −40 V, and −50 V.

Parameters extracted from the data include field-effect mobility (μ), threshold voltage (Vth), subthreshold slope (S), and the ratio of Ion/Ioff for the measured drain current. The field-effect mobility was extracted in the saturation region, where Vd>Vg−Vth. In this region, the drain current is given by the equation (see Sze in Semiconductor Devices—Physics and Technology, John Wiley & Sons (1981)):

$${I}_{d} = \frac{W}{2 L} μ {{C}_{ox} ( {V}_{g} - {V}_{th} )}^{2}$$

where W and L are the channel width and length, respectively, and Cₒₓ is the capacitance of the oxide layer, which is a function of oxide thickness and dielectric constant of the material. Given this equation, the saturation field-effect mobility was extracted from a straight-line fit to the linear portion of the √I_{d} versus Vg curve. The threshold voltage, Vₜₕ, is the x-intercept of this straight-line fit. Mobilities can also be extracted from the linear region, where Vd≦Vg−Vth. Here the drain current is given by the equation (see Sze in Semiconductor Devices—Physics and Technology, John Wiley & Sons (1981)):

$${I}_{d} = \frac{W}{L} μ {C}_{ox} [ {V}_{d} ( {V}_{g} - {V}_{th} ) - \frac{{V}_{d}^{2}}{2} ]$$

For these experiments, mobilities in the linear regime were not extracted, since this parameter is very much affected by any injection problems at the contacts. Non-linearities in the curves of I_{d} versus V_{d} at low V_{d} indicate that the performance of the device is limited by injection of charge by the contacts. In order to obtain results that are more independent of contact imperfections of a given device, the saturation mobility rather than the linear mobility was extracted as the characteristic parameter of device performance.

The log of the drain current as a function of gate voltage was plotted. Parameters extracted from the log I_{d} plot include the Iₒₙ/I_{off} ratio and the sub-threshold slope (S). The Iₒₙ/I_{off} ratio is simply the ratio of the maximum to minimum drain current, and S is the inverse of the slope of the I_{d} curve in the region over which the drain current is increasing (i.e. the device is turning on).

D. Results

The examples in Table I demonstrate that inventive devices comprising compounds based on a fused acene containing a terminal thiophene group deposited by various processes and solvents provide semiconductor films having transistor mobility and on/off ratio.

TABLE 1

| | | | | Maximum | |
|---|---|---|---|---|---|
| Ex- | | | Transistor | mobility | On/off |
| ample | Deposition | Solvent | activity | (cm²/V-s) | ratio |
| I | Vac | | yes | 0.005 | 2.0 × 10³ |
| II A | Spin coat | chlorobenzene | yes | 0.5 | 2.9 × 10³ |
| II B | Spin coat | fluorobenzene | yes | 0.08 | 2.1 × 10⁵ |
| III A | Spin coat | Isopropanol | yes | 1 × 10⁻⁴ | 9.4 × 10² |
| III B | Spin coat | chlorobenzene | yes | 6 × 10⁻⁶ | 5.5 × 10² |

It should be understood that we seek molecules that are capable of transporting holes or electrons in a transistor. Therefore, a measurable mobility of greater than 1×10⁻⁶ is an active transistor. Moreover, the mobility of an active transistor can be optimized to even higher values by process optimization.

### PARTS LIST

20 source electrode
28 substrate
30 drain electrode
44 gate electrode
56 gate dielectric
70 semiconductor

1. An article comprising, in a thin film transistor, a thin film of organic semiconductor material that comprises an acene compound comprising a linear configuration of at least three fused benzene rings, which compound has, at one end only of the linear configuration, a terminal ring that is a fused substituted or unsubstituted thiophene, which thiophene is fused on its b side to an adjacent fused benzene ring in the acene compound.

2. The article of claim 1 wherein the thin film transistor is a field effect transistor comprising a dielectric layer, a gate electrode, a source electrode and a drain electrode, wherein the dielectric layer, the gate electrode, the thin film of organic semiconductor material, the source electrode, and the drain electrode are in any sequence as long as the gate electrode and the thin film of organic semiconductor material both contact the dielectric layer, and the source electrode and the drain electrode both contact the thin film of the organic semiconductor material.

3. The article of claim 1 wherein the thin film of organic semiconductor material is capable of exhibiting electron mobility greater than 0.01 cm²/Vs.

4. The article of claim 1, wherein the thin film of organic semiconductor material comprises an acene compound represented by the following structure: 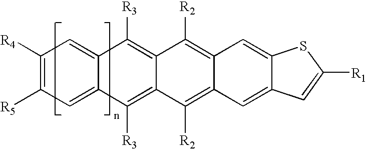
wherein n is an integer from 0 to 5, where at least one of R₂ or R₃ is hydrogen and wherein R₂ or R₃ is independently selected from hydrogen, branched or unbranched alkane having 2 to 18 carbon atoms, a branched or unbranched alkyl alcohol having 1 to 18 carbon atoms, a branched or unbranched alkene having 2 to 18 carbon atoms, a branched or unbranched alkyne having 2 to 18 carbon atoms, an aryl or heteroaryl having 4 to 8 carbon atoms, an alkylaryl or alkyl-heteroaryl having 5 to 32 carbon atoms, which groups except hydrogen can be substituted or unsubstituted;
R₁, R₄, and R₅ are independently selected from organic or inorganic groups that do not adversely affect the p-type semiconductor properties of the material.

5. The article of claim 4 wherein R₂ and R₃ are both hydrogen.

6. The article of claim 4 wherein only one of R₂ and R₃ are hydrogen.

7. The article of claim 4 wherein R₄ and R₅ are hydrogen.

8. The article of claim 4 wherein R₁ is selected from hydrogen, halogen atoms, substituted and unsubstituted alkyl groups, alkoxy groups, and thioalkoxy groups or an organic group containing an alcohol, phenol, thiol, carboxylic acid, amide, or carbamate functionality to achieve a hydrogen bonding interaction, or an alkyl, aryl, perfluoroalkyl, perfluoroaryl, or siloxane functionality to achieve hydrophobic interactions, or a trichlorosilane, trialkoxysilane, acid chloride, or N-hydroxysuccinimide ester of a carboxylic acid to achieve reaction.

9. The article of claim 4 wherein R₄ and R₅ are independently selected from the group consisting of electron-donating groups, halogen atoms, hydrogen atoms, and combinations thereof.

10. The article of claim 4 wherein n is 1 or 2 and R₄ and R₅ are independently selected from the group consisting of hydrogen, halogen atoms, and substituted and unsubstituted alkyl groups, alkoxy groups, and thioalkoxy groups, and combinations thereof.

11. The article of claim 4 wherein R₁ is a short-chain C1 to 6 alkyl group substituted with a functionality comprising at least one hydroxyl or carbonyl group.

12. The article of claim 4 wherein one of R₂ and R₃ is hydrogen and the other is selected from the group consisting of:
—C≡C—R₆ where R₆=H or alkyl or aryl
—C≡C—Si(R₇)₃ where R₇=alkyl

13. The article of claim 1, wherein the thin film of organic semiconductor material comprises a compound that is an acene compound represented by the following structure: 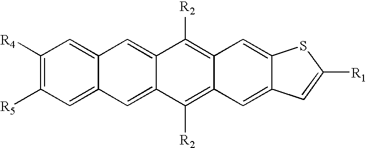
wherein R₂, R₄, R₅ and R₁ are as define above.

14. The article of claim 13 wherein R₂, R₄ and R₅ are hydrogen.

15. The article of claim 13 wherein R₂ is not hydrogen.

16. The article of claim 4, wherein the thin film of organic semiconductor material comprises an acene compound represented by the following structure: 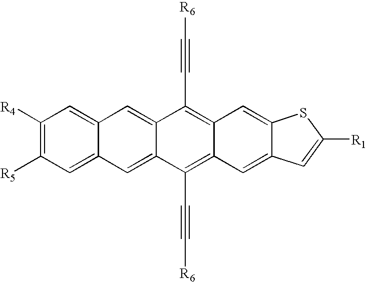
wherein both R₆ groups are the same, R₁, R₄, and R₅ are as defined previously, and R₆ is a branched or unbranched alkane having 2 to 18 carbon atoms, a branched or unbranched alkyl alcohol having 1 to 18 carbon atoms, an aryl or heteroaryl having 4 to 8 carbon atoms, an alkylaryl or alkyl-heteroaryl having 5 to 32 carbon atoms or a hydrogen, or a Si(R₇)₃ group where the R₇ group is independently a branched or unbranched alkane having 1 to 10 carbon atoms, a branched or unbranched alkyl alcohol having 1 to 10 carbon atoms, or a branched or unbranched alkene having 2 to 10 carbon atoms.

17. The article of claim 16 wherein R₄ and R₅ are hydrogen.

18. The article of claim 1, wherein the thin film transistor has an on/off ratio of a source/drain current of at least 10⁴.

19. The article of claim 2, wherein the gate electrode is adapted for controlling, by means of a voltage applied to the gate electrode, a current between the source and drain electrodes through said organic semiconductor material and wherein the gate dielectric comprises an inorganic or organic electrically insulating material.

20. The article of claim 1 wherein the thin film transistor further comprises a non-participating support that is optionally flexible.

21. The article of claim 2 wherein the source, drain, and gate electrodes each independently comprising a material selected from doped silicon, metal, and a conducting polymer.

22. An electronic device selected from the group consisting of integrated circuits, active-matrix display, and solar cells comprising a multiplicity of thin film transistors according to claim 1.

23. The electronic device of claim 22 wherein the multiplicity of the thin film transistors is on a non-participating support that is optionally flexible.

24. A process for fabricating a thin film semiconductor device, comprising, not necessarily in the following order, the steps of:
(a) depositing, onto a substrate, a thin film of organic semiconductor material comprising an acene compound comprising a linear configuration of at least three fused benzene rings, which compound has, at one end only of the linear configuration, a terminal ring that is a fused substituted or unsubstituted thiophene, fused on its b side to an adjacent fused benzene ring in the acene compound, such that the thin film of organic semiconductor material exhibits a field effect electron mobility that is greater than 0.01 cm²/Vs;
(b) forming a spaced apart source electrode and drain electrode, wherein the source electrode and the drain electrode are separated by, and electrically connected with, the n-channel semiconductor film; and
(c) forming a gate electrode spaced apart from the semiconductor material.

25. The process of claim 21, wherein the compound is deposited on the substrate by sublimation or by solution-phase deposition and wherein the substrate has a temperature of no more than 100° C. during deposition.

26. The process of claim 21 comprising, not necessarily in order, the following steps:
(a) providing a support;
(b) providing a gate electrode material over the substrate;
(c) providing a gate dielectric over the gate electrode material;
(d) depositing the thin film of organic semiconductor material over the gate dielectric:
(e) providing a source electrode and a drain electrode contiguous to the thin film of organic semiconductor material.

27. The process of claim 26 wherein the support is flexible.

28. The process of claim 26 carried out in its entirety below a peak temperature of 100° C.

29. An integrated circuit comprising a plurality of thin film transistors made by the process of claim 26.

30. A compound having the following structure: 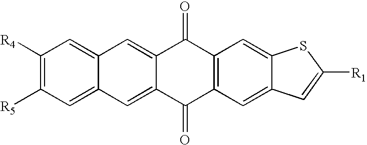
wherein R₁, R₄, and R₅ is independently selected from hydrogen, fluorine, and organic groups.
